(19) United States
(12) Patent Application Publication
(10) Pub. No.: US 2022/0111193 A1
(43) Pub. Date: Apr. 14, 2022

(54) HEMOSTATIC VALVE EXTENSION

(71) Applicant: Duke University, Durham, NC (US)

(72) Inventors: Charles Geddie, Durham, NC (US); Kathleen Carroll, Salem, MA (US); Shweta Shrikant, Durham, NC (US); Manesh Patel, Durham, NC (US); Guillaume Marquis-Gravel, Mont-Royal (CA)

(21) Appl. No.: 17/497,602

(22) Filed: Oct. 8, 2021

Related U.S. Application Data
(60) Provisional application No. 63/089,062, filed on Oct. 8, 2020.

(51) Int. Cl. A61M 39/06

(52) U.S. Cl. CPC A61M 39/06; A61M 2039/062

(57) ABSTRACT

A hemostatic valve extension is provided that allows a clinician to perform interventional procedures from any location around the patient rather than being limited to a particular side, often the right side. The extension provides for a hemostatic connector, flexible tube, and a replacement hemostatic valve that allows the original hemostatic valve, which can be, for example, part of an introducer sheath, to be bypassed and the replacement hemostatic valve to be used to perform the medical procedure from a desired location of the clinician. The extension can include a distal end connecting element that is configured to be used with a plurality of hemostatic valve types such that the connector can be used with different sized hemostatic valves without having to swap parts or components on the connector.

### CROSS REFERENCE TO RELATED APPLICATION

The present disclosure claims priority to and the benefit of U.S. Provisional Patent Application No. 63/089,062, entitled “HEMOSTATIC VALVE EXTENSION,” filed on Oct. 8, 2020, the disclosure of which is incorporated by reference herein in its entirety.

### FIELD

The present disclosure relates to devices and methods for allowing an operator to easily perform an interventional medical procedure (e.g., a catheterization) from any side of the body, and more particularly relates to a hemostatic extension that can be coupled to existing hemostatic valves to allow for the interventional medical procedure to be performed from any side of the body.

### BACKGROUND

The minimally invasive nature of interventional medical procedures make them a popular and increasingly important option for use with respect to radiology-, cardiology-, and endovascular surgical neuroradiology-based procedures, among other procedures. Interventional medical procedures can be used for imaging and catheterization to diagnose and treat vascular issues in the body, and can include injecting arteries with dye, visualizing these via x-ray, and opening up blockages. The procedures can also be used in conjunction with removing fluid (e.g., blood, urine) from the body. Interventional medical procedures allow for safer and better ways to treat various vascular and cardio-related diseases, and safer and better surgical procedures more generally.

Traditionally, catheterizations and other interventional medical procedures are performed with the operator being positioned on a right side of a patient, and thus rooms or labs where the procedures are performed are set-up for this one-sided approach. For catheterizations, a lead shield is typically placed between the operator and an x-ray machine. The operator manipulates the catheters outside the body at the level of the sheath, where the operator's left hand is on the hub of the sheath, and his/her right hand feeds a catheter through the sheath. The operator both pushes and pulls the catheter through the hub, thereby torqueing the catheter.

A challenge arises when a procedure must be performed on the left or opposite side of the patient, because of the particular treatment that is needed and/or because of the handedness (i.e., left-handed or right-handed) of the operator. In this case, the operator must bend over for the duration of the procedure, which can be up to a several hours for complex procedures. This over-extended position can lead to a variety of ergonomic issues, such as back, neck, and shoulder pain. A recent study showed that over 40% of interventional cardiologists report spine problems. Further, by virtue of having to bend over and otherwise contort his/her body, the performance of the procedure by the operator may be negatively impacted.

Moreover, in addition to discomfort and possible decreased performance in carrying his/her duties out, interventional cardiologists spend their career in the catheterization laboratory, exposing themselves to low-grade ionizing radiation. Even with the added protection of leaded shields, drapes, aprons, and glasses, interventional cardiologists are exposed to higher radiation than other specialists. If an operator needs to bend over to access the left side of the patient, they may be bypassing the leaded shield, thus exposing their hands, upper thorax, and sometimes head to radiation.

Accordingly, there is an ongoing need for improved methods and systems for interventional medical procedures, such as catheterizations and other hemostatic valve procedures, that presently require an operator to reach across the patient's body to perform the procedure.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure provides for hemostatic valve extensions that can be used in conjunction with existing introducer sheaths and/or hemostatic valves to extend a range of locations from where a medical procedure can be performed. The extensions include a versatile hemostatic connector, a flexible tube, and a replacement hemostatic valve. The hemostatic connector is versatile in that it is configured in a manner that enables it to be used with a variety of hemostatic valves having a variety of configurations. More particularly, a distal end connecting element is adaptable for use with different sized hemostatic valves (and can be adapted for different shapes as well) by virtue of having a plurality of connection points, e.g. barbs, having different sizes for use with the different sized existing hemostatic valves. A proximal end connecting element of the extension can be coupled with the flexible tube, thereby creating a pathway for fluid to flow through the original/existing hemostatic valve, through the hemostatic connector, through the tube, and to a second hemostatic valve located on the other side of the flexible tube. Likewise, fluid can flow in the other direction along the same created path, so from the second hemostatic valve, through the flexible tube, through the hemostatic connector, through original/existing hemostatic valve, and into a patient's body, such as when injecting saline via a flush line as provided for herein and otherwise known to those skilled in the art. Either way, the use of the extension effectively moves the location of where the procedure will be performed from the original/existing hemostatic valve to the location of the second hemostatic valve.

One embodiment of a hemostatic valve extension includes a hemostatic connector, a flexible tube, and a replacement hemostatic valve. The hemostatic connector has a body, a distal end connecting element, a proximal end connecting element, and an internal passageway that extends through the body from a distal terminal end of the hemostatic connector to a proximal terminal end of the hemostatic connector. A distal end of the flexible tube is coupled to the proximal end connecting element and a proximal end of the flexible tube is coupled to the replacement hemostatic valve. The flexible tube is in fluid communication with the internal passageway of the hemostatic connector and the replacement hemostatic valve. The distal end connecting element includes a rotation-independent mating feature, which is configured to couple to an existing hemostatic valve without rotating either the distal end connecting element or the existing hemostatic valve.

The rotation-independent mating feature can include at least one male barb that is disposed on an outer surface of the distal end connecting element. In some embodiments, the at least one male barb includes at least two male barbs. A maximum diameter of a first such barb can be different than a maximum diameter of a second such barb. The outer surface of the distal connecting element can be tapered between the two male barbs. Alternatively, or additionally, the outer surface of the distal connecting element can be tapered between the distal terminal end of the hemostatic connector and the first male barb.

The maximum diameter of each of the first and second male barbs can be approximately in the range of about 3 millimeters to about 6 millimeters. An axial length of the first male barb can be approximately in the range of about 0.5 millimeters to about 7 millimeters. In some embodiments, the maximum diameter of the first male barb can be approximately 3.9 millimeters and the maximum diameter of the second male barb can be approximately 4.2 millimeters. In some embodiments, an axial length of the first male barb can be approximately 4.3 millimeters. Other dimensions related to the barb(s), and the extension and its related components more generally, are possible.

A proximal portion of the body can include one or more ergonomic features disposed on it to provide a gripping surface. The body can include a flange disposed between the distal end connecting element and a proximal portion of the body. In some embodiments, the flexible tube and the hemostatic connector can form a single part.

One embodiment of a hemostatic connector includes a body, a distal end connecting element, a proximal end connecting element, and an internal passageway that extends through the body from a distal terminal end of the hemostatic connector to the proximal terminal end surface of the hemostatic connector. The distal end connecting element includes at least two male barbs that are disposed on an outer surface of the distal end connecting element. A maximum diameter of a first male barb of the at least two male barbs is different than a maximum diameter of a second male barb of the at least two male barbs.

The outer surface of the distal connecting element can be tapered between the first male barb and the second male barb. Alternatively, or additionally, the outer surface of the distal connecting element can be tapered between the distal terminal end of the hemostatic connector and the first male barb.

The maximum diameter of each of the first and second male barbs can be approximately in the range of about 3 millimeters to about 6 millimeters. An axial length of the first male barb can be approximately in the range of about 0.5 millimeters to about 7 millimeters. In some embodiments, the maximum diameter of the first male barb can be approximately 3.9 millimeters and the maximum diameter of the second male barb can be approximately 4.2 millimeters. In some embodiments, an axial length of the first male barb can be approximately 4.3 millimeters. Other dimensions related to the barb(s), and the extension and its related components more generally, are possible.

A proximal portion of the body can include one or more ergonomic features disposed on it to provide a gripping surface. The body can include a flange disposed between the distal end connecting element and a proximal portion of the body. In some embodiments, the hemostatic connector can include a flexible tube coupled to the proximal end connecting element. The flexible tube can be in fluid communication with the internal passageway of the hemostatic connector. In at least some such embodiments, the flexible tube and the hemostatic connector can form a single part.

The at least two barbs can be rotation-independent mating features. Such a configuration can allow the hemostatic connector to couple to an existing hemostatic valve without rotating either the distal end connecting element or the existing hemostatic valve.

A method of performing an interventional medical procedure includes coupling a hemostatic connector to a first hemostatic valve disposed in a patient without rotating either the hemostatic connector or the first hemostatic valve, and performing one or more interventional medical procedures from a location that is on an opposite side of the patient than where the first hemostatic valve is located. The hemostatic connector is coupled to a flexible tube and the flexible tube is coupled to a second hemostatic valve. The hemostatic connector, the flexible tube, and the second hemostatic valve are in fluid communication with each other. The action of coupling the hemostatic connector to the first hemostatic valve allows the first hemostatic valve to be bypassed and the second hemostatic valve to be operable to perform one or more medical procedures.

In at least some embodiments, the action of coupling a hemostatic connector to a first hemostatic valve can include pushing a distal end connecting element of the hemostatic connector into the first hemostatic valve, forming a seal between them. The distal end connecting element can include at least two male barbs disposed on its outer surface. A maximum diameter of a first such barb can be different than a maximum diameter of a second such barb, thereby enabling the hemostatic connector to be compatible with multiple sizes of hemostatic valves that can be the first hemostatic valve.

A number of interventional medical procedures can be performed. Such procedures include, but are not limited to, arterial procedures that use percutaneous introduction of intravascular devices, cardiology procedures, procedures involving Extracorporeal Membrane Oxygenation Circuits, and/or catheterizing.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments:

FIG. 1 is a side view of one exemplary embodiment of a hemostatic valve extension;

FIG. 2A is a side perspective view of a hemostatic connector of the hemostatic valve extension of FIG. 1

FIG. 2B is a cross-sectional view of the hemostatic connector of FIG. 2A taken along line B-B (dimensions illustrated in millimeters, the dimensions being non-limiting);

FIG. 3A is a perspective view of the hemostatic connector of FIG. 2A having a flexible tube of the hemostatic valve extension of FIG. 1 mated thereto;

FIG. 3B is a side view of the hemostatic connector and flexible tube of FIG. 3A;

FIG. 3C is a cross-sectional view of the hemostatic connector and the flexible tube of FIG. 3B taken along line C-C;

FIG. 4 is a side view of another exemplary embodiment of a hemostatic valve extension, the hemostatic valve extension being coupled to an existing hemostatic valve;

FIG. 5A is a progressive series of cross-sectional side views of the hemostatic connector of FIG. 2A being inserted into an existing hemostatic valve; and

FIG. 5B is a concurrent progressive series of cross-sectional perspective views of the progressive series of cross-sectional side views of FIG. 5A.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Articles “a” and “an” are used herein to refer to one or to more than one (i.e. at least one) of the grammatical object of the article. By way of example, “an element” means at least one element and can include more than one element. “About” is used to provide flexibility to a numerical range endpoint by providing that a given value may be “slightly above” or “slightly below” the endpoint without affecting the desired result. The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

Moreover, the present disclosure also contemplates that in some embodiments, any feature or combination of features set forth herein can be excluded or omitted. To illustrate, if the specification states that a device or apparatus comprises components A, B, and C, it is specifically intended that any of A, B, or C, or a combination thereof, can be omitted and disclaimed singularly or in any combination. Similarly, to the extent features or steps are described herein as being a “first feature” or “first step,” or a “second feature” or “second step,” such numerical ordering is generally arbitrary, and thus such numbering can be interchangeable. Moreover, a person skilled in the art will appreciate that not all of the method steps disclosed herein are required, and, in view of the present disclosure, will understand how modifications can be made to each step, the order of the steps, the limitation of certain steps, etc. without departing from the spirit of the present disclosure while still achieving the desired goals.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as approximately in the range of about 1% to about 50%, it is intended that values such approximately in the range of about 2% to about 40%, approximately in the range of about 10% to about 30%, or approximately in the range of about 1% to about 3%, etc., are expressly enumerated in this specification. These are only examples of what is specifically intended, and all possible combinations of numerical values between and including the lowest value and the highest value enumerated are to be considered to be expressly stated in this disclosure, as are values slightly above and/or slightly below those ranges at least in instances in which the term “about” is used. To the extent that linear or circular dimensions are used herein, such dimensions are not intended to limit the types of shapes that can be used in conjunction with the same. A person skilled in the art will recognize that an equivalent to such linear and circular dimensions can easily be determined for any geometric shape.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human who is undergoing a medical procedure with systems and methods as prescribed herein. Similarly, the terms “operator,” “clinician,” and other similar terms are used to refer to any person, or non-person (e.g., robot, computer, etc.), performing the described or recited procedure, or one or more steps thereof.

A number of terms may be used throughout the disclosure interchangeably but will be understood by a person skilled in the art. By way of non-limiting example, the terms “hemostasis” and “hemostatic,” at least when used as an adjective, may be used interchangeably. Terms like “tube,” “tubing,” and “conduit” provide another non-limiting example of interchangeable terms. Unless noted otherwise, the term “distal” is intended to refer to a location, side, or portion that is closer to a patient and the term “proximal” is intended to refer to a location, side, or portion that is closer to an operator. Still further, use of the term “end,” such as in the context of describing a “distal end” or a “proximal end,” does not require that the described feature be located at a terminal end, but rather describes a general vicinity of the location. This may be considered to be interchangeable in at least some respects with the term “portion,” such as a “distal portion” or a “proximal portion.” The foregoing notwithstanding, illustrated embodiments may demonstrate certain features that may be located at a terminal end without actually describing it as being at a “terminal end.”

Unless otherwise defined, all technical terms used herein have the same meaning as commonly understood by one of ordinary skill in the art to which this disclosure belongs. Additionally, like-numbered components across embodiments generally have similar features unless otherwise stated or a person skilled in the art would appreciate differences based on the present disclosure and his/her knowledge. Accordingly, aspects and features of every embodiment may not be described with respect to each embodiment, but those aspects and features are applicable to the various embodiments unless statements or understandings are to the contrary.

One aspect of the present disclosure provides a solution to the challenges of performing interventional procedures, such as catheterizations, of a subject on the side of the patient that is opposite from where the clinician is standing. Currently, the clinician must lean over the patient to perform the functions, which can lead to fatigue and/or pain, can negatively impact the clinician's performance of the procedure, and can require the clinician to potentially be exposed to medical radiation (or more medical radiation). With the systems and methods disclosed herein, the clinician can more comfortably and more safely perform these tasks. This is accomplished by moving the functional components nearer to the clinician.

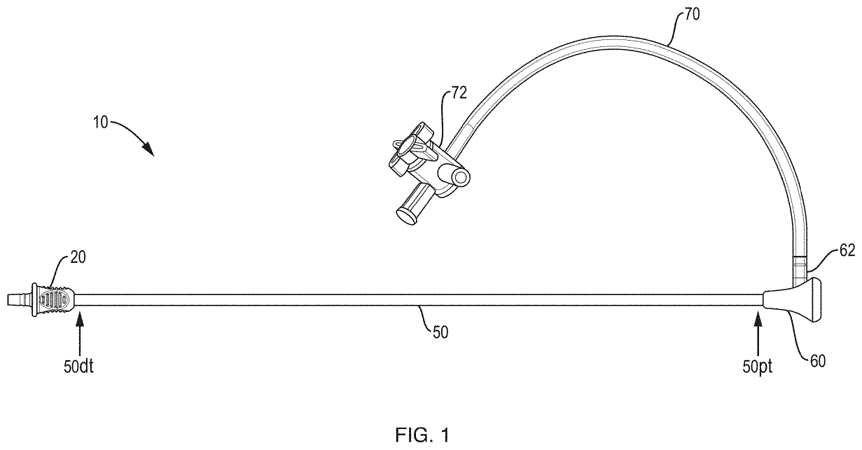

FIG. 1 is an example embodiment of a hemostatic valve extension 10. The extension includes a hemostatic connecting device or connector 20, a flexible tube 50, and a hemostatic valve 60, also referred to as a “second” or “replacement” hemostatic valve. A first terminal end 50dt of the flexible tube 50 is coupled to the hemostatic connector 20 and a second, opposite terminal end 50pt of the flexible tube 50 is coupled to the hemostatic valve 60. Also shown is a flush line 70, or side tube, with a control valve 72 (e.g., stopcock) coupled to the hemostatic valve 60, the flush line 70 being configured to control fluid flow through the hemostatic valve 60 (e.g., by way of the stopcock), and thus the flexible tube 50 and hemostatic connector 20. In the illustrated embodiment, the flush line 70 is coupled to a port 62 of the hemostatic valve 60. The hemostatic connecting device 20 is configured to attach to an existing (or “first”) hemostatic valve on an opposite end from an end that is coupled to the flexible tube 50, such as at an inlet port or opening of the existing hemostatic valve. The first hemostatic valve can become a pass-through connection, and the flexible tube 50 can extended across or over the patient. The operator then uses the second hemostatic valve 60 to perform the prescribed clinical functions that would normally occur at the first hemostatic valve. This allows the operator to stand at a more desirable location, i.e., at a position that affords a more desirable and/or normal posture, such as if the procedure were being performed on the near side of the patient's body.

While the embodiment illustrated in FIG. 1 is described as an extension, it can be considered a system or device that comprises one or more of the components illustrated (e.g., the hemostatic connector 20, the flexible tube 50, the hemostatic valve 60, the flush line 70). Further, in some instances the extension 10, or the system or device, may be considered only some portion of the illustrated components, such as just the hemostatic connector 20 or a combination of the hemostatic connector 20 and the flexible tube 50. A person skilled in the art will appreciate that the various components of the described extension or system 10 can be manufactured, packaged, sold, and/or otherwise distributed separately or in any combination, including with other components or devices not disclosed herein but known to those skilled the art as being able to be used in conjunction with extensions, systems, or devices, or components thereof, of the present disclosure.

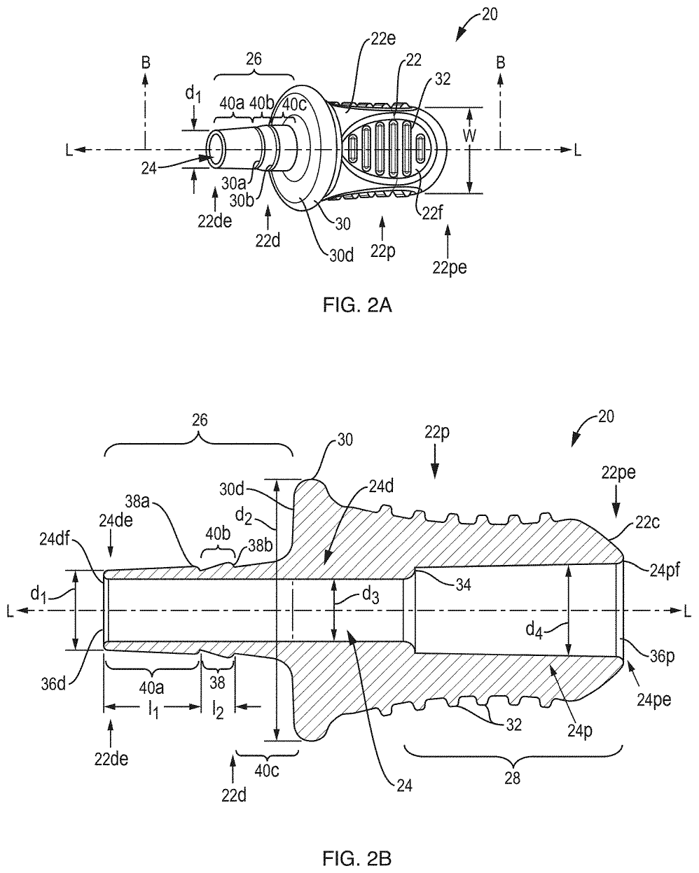

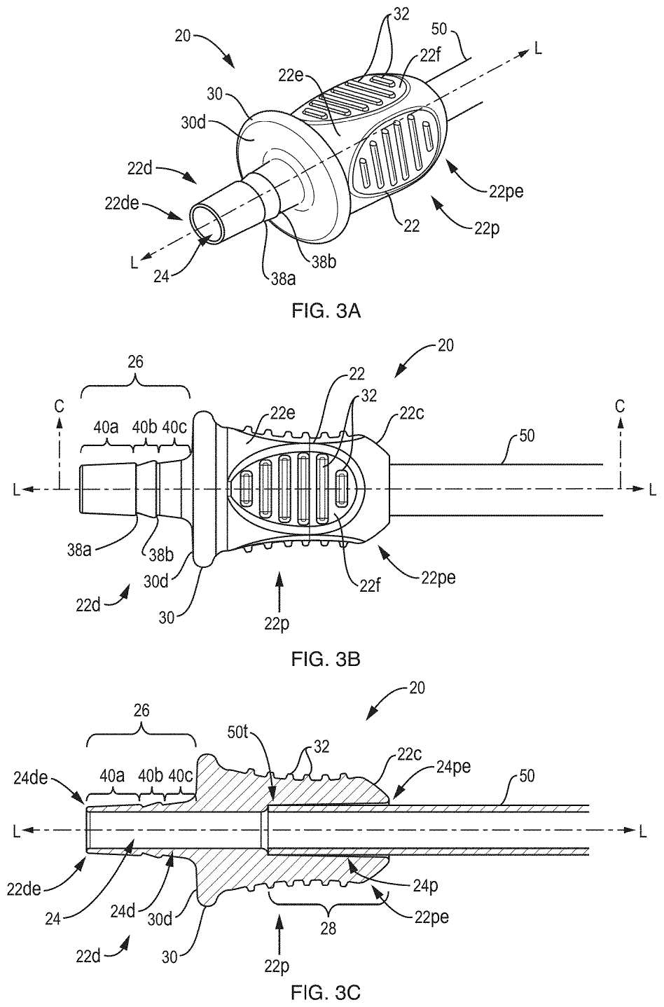

The hemostatic connecting device or connector 20 is illustrated in more detail in FIGS. 2A and 2B and FIGS. 3A-3C. The connector 20 has a body 22, an internal passageway 24, and both a distal end connecting element 26 and a proximal end connecting element 28 located at opposed distal and proximal ends 22de, 22pe of the body 22. The body 22 provides the structure of the connector 20, and can be engineered or otherwise formulated for particular uses or purposes, at least some of which are described in greater detail below, but others of which can be understood by a person skilled in the art. In the illustrated embodiment, a proximal portion 22p of the body 22 has a generally rectangular prism shape with filleted edges 22e while a distal portion 22d of the body 22 has a generally cylindrical shape, with a width w of the proximal portion 22p being greater than a diameter d₁ of the distal portion 22d. This is because the proximal portion 22p is designed to be grabbed or held by the operator while the distal portion 22d is designed to be connected to an existing hemostatic valve to be used in an interventional procedure, and thus should be less invasive and sized and shaped to work well with the existing hemostatic valve. In some non-limiting embodiments, the width w across a substantially flat surface of the proximal portion 22p of the body 22 is approximately in the range of about 6 millimeters to about 12 millimeters. In FIG. 2B, the width w at one location of the proximal portion 22p is 7.5 millimeters, although as described herein, that width may change depending upon the location at which it is measured. This can be due, for example, due to a tapered configuration in which, as shown, the proximal portion 22p tapers for a portion as it extends towards the proximal end 22pe of the body 22, and then gets larger again as it approaches the proximal end 22pe before a chamfer-shaped or rounded-shaped region 22c that decreased in width.

An optional flange 30 can be disposed between the proximal and distal portions 22p, 22d of the body 22, the illustrated flange 30 having a diameter d₂ that is even greater than the width w of the proximal portion 22p of the body 22. The flange 30 can serve as a stop surface such that a distal-facing surface 30d of the flange 30 engages a surface associated with the hemostatic valve to which the connector 20 is being inserted. The flange 30 can also aid the operator in preventing the operator's hand from slipping distally towards the body, provide an out-of-plane (e.g., non-axial) gripping surface for helping the operator secure the connection to an existing hemostatic valve, and/or otherwise help the operator identify the location of the connector 20 for gripping. As more clearly illustrated in FIG. 2B, the proximal portion 22p of the body 22 can be tapered such that the illustrated width w increases in the direction of the flange 30 for at least a portion of the length of the body 22. Further, the proximal portion 22p can optionally comprise ergonomic features to improve gripping of the connector 20. As shown, this can include a region with a relatively larger volume for comfortable handling, as well as one or more flat surfaces 22f and/or the tapered configuration of the proximal portion 22p. Additionally, the proximal portion 22p can include gripping features 32, such as ridges, knurling, texturization, etc., to facilitate pressing the connector 20 into the first hemostatic valve.

Disposed within the body 22 is the internal passageway 24, more clearly illustrated in FIG. 2B. The internal passageway 24 extends from the proximal end 22pe to the distal end 22de of the body 22 and can be configured in a variety of ways. For example, in some instances the passageway 24 can be configured to be more universal so that it can receive a variety of medical instruments (e.g., catheters and related components of various sizes) therethrough, while in other instances the passageway 24 may be configured to receive one or more particular types, shapes, and/or designs of medical instruments therethrough. As shown, a distal portion 24d of the passageway 24 has a smaller diameter d₃ than a diameter d₄ of the proximal portion 24p of the passageway 24. Such a configuration can be beneficial when a medical instrument being inserted therethrough utilizes a delivery tool that has a wider diameter than the medical instrument itself. The transition from the smaller diameter d₃ of the distal portion 24d to the larger diameter d₄ of the proximal portion 24p can provide a convenient stop surface 34 that prevents the delivery tool from pushing into the distal portion 24d of the passageway 24. Alternatively, or additionally, the internal passageway 24 can be configured in a manner that assists in coupling the flexible tube 50 to the hemostatic connector 20. For example, as shown in FIG. 3C, the stop surface 34 can be utilized in conjunction with coupling the flexible tube 50 to the hemostatic connector 20.

Disposed at opposed ends 24de, 24pe of the internal passageway 24 can be sealing surfaces 36d, 36p, respectively, designed to form a seal between instruments inserted into the internal passageway 24 and the instrument being inserted. Accordingly, as shown, a lead-in chamfered-area and/or flanged portion 24pf at the proximal end 24pe of the internal passageway 24 can aid in providing such a seal, as can a lead-in chamfered-area or flanged portion 24df at the distal end 24de of the internal passageway 24.

In the illustrated embodiment, the distal end connecting (or coupling) element 26 includes one or more male barbs 38. As shown, there are two male barbs 38a, 38b, with each barb being specifically designed to connect to a variety of different conventional hemostatic valves. The illustrated barbs 38 are formed as rings, having a substantially similar contour and configuration across a 360 degree rotation about the surface of the body 22, the rotation being about a longitudinal axis L extending through the internal passageway 24. As described herein, the tapered configuration between the barbs 38 results in a changing diameter d₁ of the distal end connecting element 26 along the axial length, but as at least FIG. 2A and the related cross-section of FIG. 2B illustrate, the barbs 38 maintain a diameter across a cross-section or slice of the distal end connecting element 26. That is, each barb 38a, 38b forms a ring around the longitudinal axis L, with the ring not moving along the axial length of the body 22. This is in contrast to a thread, which moves along the axial length to allow for rotation of the device to effect sealing and locking. The securement of the connector 20 to the first hemostasis valve is controlled, at least in part, by the diameter and the length of the barbs 38.

More specifically, in the non-limiting example hemostatic connector 20 illustrated in FIG. 2B, the distal end connecting element 26 has a plurality of barbs 38 formed or otherwise disposed on an outer surface of the distal end connecting element 26. The barbs 38 can increase in diameter as they get closer to the proximal portion 22p of the body 22 (i.e., in the proximal direction). For example, the distal end connecting element 26 can have a first, outermost or distal-most barb 38a with a maximum diameter of approximately 3.9 millimeters, and an innermost or proximal-most barb 38b with a maximum diameter of approximately 4.2 millimeters, the maximum diameter being the largest diameter formed by the barb 38a, 38b, i.e., the portion of the barb furthest radially away from the longitudinal axis L. These dimensions allow the connector 20 to engage with the hemostatic valve of the Merit Prelude, Oscor Adelante, and Teleflex Arrow sheath sets at the outermost barb 38a, and with the Terumo Pinnacle hemostatic valve at the innermost barb 38b. It is also possible to have additional barbs of any suitable size and/or position for attaching to the first hemostatic valve. More generally, a maximum diameter of the one or more barbs 38 can be approximately in the range of about 3 millimeters to about 5 millimeters, the values being based, at least in part, on the dimensions of the hemostatic valves with which the extension 10 is going to be used. It is likewise possible to include only a single barb. As shown, the outer surface of the distal portion 22d of the body 22 tapers distally such that the diameter d₁ increases from the distal end 22de and towards the respective barbs 38, with the diameter d₁ decreasing again between barbs 38 before an additional taper. Accordingly, as shown, there is a first tapered portion 40a of the outer surface of the distal end connecting element 26 between the distal end 22de and the first barb 38a, a second tapered portion 40b of the outer surface of the distal end connecting element 26 between the first barb 38a and the second barb 38b, and a third tapered portion 40c of the outer surface of the distal end connecting element 26 between the second barb 38b and the flange 30. Similar to other aspects of the hemostatic connector 20, although the illustrated embodiment shows the distal end connecting element 26 having a generally cylindrical shape, other shapes and configurations are possible without departing from the spirit of the present disclosure. Of more importance is that the distal end connecting element 26, and thus the barb(s) 38 of the same, be configured in a manner such that engagement with a first hemostatic valve results in a sealed configuration that limits or otherwise prevents fluid leakage.

In some embodiments, an outermost barb length l₁ (i.e., longitudinal or axial length) can be configured so that dislodgement that may be caused by movement of the flexible tube 50 is minimized. For example, the barb(s) 38 can be designed to fit within the chamber of a hemostatic valve, but have sufficient length to prevent separation of the connecting device 20 if tension, shear, or bending forces are applied to the assembly or extension 10. In some embodiments, this axial length l₁ is approximately 4.3 millimeters. More generally, the axial length l₁ can be approximately in the range of about 3 millimeters to about 6 millimeters. Likewise, an innermost barb length l₂ can be optimized so that when fully inserted, the base of the hemostatic connector 20, e.g., the distal-facing surface 30d of the flange 30, is flush or substantially flush against the existing hemostatic valve, minimizing or eliminating leaks. The axial length l₂ of the innermost barb can be approximately in the range of about 0.5 millimeters to about 7 millimeters, and in the illustrated embodiment it is approximately 1.5 millimeters.

The distal end connecting element 26 can be configured to provide tactile and/or audible feedback to the operator to confirm that hemostatic connector 20 has formed a properly sealed connection with the first hemostatic valve (or other component with which the hemostatic connector 20 is connected). The tactile feedback can be a result of the tapered configuration that allows the distal end connecting element 26 to progressively secure itself to the first hemostatic valve with the barb(s) 38 being configured to tightly grip the first hemostatic valve in a manner that provides the tactile feedback to the user that the sealed connection has been formed. An attempt to separate the connector 20 from the first hemostatic valve can be more difficult, thus further confirming the connection has been properly made. Accordingly, the operator knows a sealed connection is in place. Audible feedback can be formed by virtue of the materials and/or contours associated with the barb(s) 38 vis-à-vis the portion of the first hemostatic valve to which the distal end connecting element 26 is designed to be coupled. A sound can be generated by virtue of the connection between the barb(s) 38 and the first hemostatic valve being formed, thereby informing the operator that a sealed connection is in place. The feedback, whether tactile or audible, informs the operator that the extension 10 can be used to perform the procedure at the desired location of the operator with respect to the patient and other equipment in the room.

Although in the illustrated embodiment the distal end connecting element 26 is illustrated and described as one or more barbs 38, a person skilled in the art will appreciate other mating features that can be used in lieu of or in addition to barbs. Some non-limiting examples include a tapered end capable of providing a press fit or friction fit (this could be without barbs or other protruding features), one or more protrusions having similar capabilities as the one or more barbs, one or more annular rings, or a plurality of cantilever arms that each have one or more ledges configured to bend inwards during insertion and secure in place once sufficiently passed through the existing hemostatic valve, among other mating features known or otherwise derivable from the present disclosures. As described in greater detail below, the mating features of the distal end connecting element are typically rotation-independent.

FIG. 2B also shows further details of the proximal end connecting element 28. The proximal end connecting element 28, which is configured to couple to, mate, or otherwise connect to the flexible tube 50, as shown in FIGS. 3A-3C, can be formed in any suitable manner, such as an internal or external fitting. The coupling formed between the proximal end connecting element 28 and the flexible tube 50 can be done in a variety of ways and generally in a manner that minimizes or eliminates leakage therebetween. This includes, but is not limited to using mechanical fits (e.g., press-fits, snap-fits, male-female receivers/adapters), adhesives (e.g., glue), welding, and other techniques known to those skilled in the art for creating mating two components together while maintaining a seal therebetween. In some embodiments, the hemostatic connector 20 and the flexible tubing 50 can be a single part, e.g., monolithic, such that the proximal end connecting element 28 is formed in unison with the flexible tubing 50. In the illustrated embodiment, the proximal end connecting element 28 is a female press-fit connection configured to accommodate standard medical tubing. The lead-in chamfered area 24pf can facilitate easy connection. In some embodiments, the hemostatic connector 20 is optionally made of a monolithic material that has some flexibility, such as PC or ABS plastic. Other materials, or combinations of materials, can be used to form the hemostatic connector 20.

The flexible tubing 50, or flexible extension tube or conduit, is configured to be attached to the proximal end connecting element 28 at the first, distal end 50dt, and to the second hemostatic valve 60 at the second, proximal end 50pt to allow fluid flow therethrough. The flexible extension tube 50 can be selected, at least in part, according to the prescribed usage, taking into account variables such as catheter length, patient size, etc. In some embodiments, the length is approximately in the range of about 10 centimeters to about 30.5 centimeters. Further, multiple tubing sections can be provided in various lengths in a kit of parts for the system. Such a kit can likewise include a plurality of hemostatic connectors and/or a plurality of hemostatic valves and/or a plurality of flush lines, as well as related components. In some instances it is important for the operator to know the length of the tubing 50. Thus, the tubing 50 can also have fiducial markings or labels (not shown) indicating the length.

The flexible extension tube 50 can be formed from a kink-resistant material with a smooth inner lumen to facilitate movement and transfer of interventional devices through the extended pathway. Some non-limiting example materials include a multi-layered reinforced tube (e.g., PEBA outer jacket, with reinforced SS braids, inner FEP/PTFE liner), a co-extrusion (e.g., PEBA, low friction liner), and a single extrusion (e.g., Lubricious Pebax®).

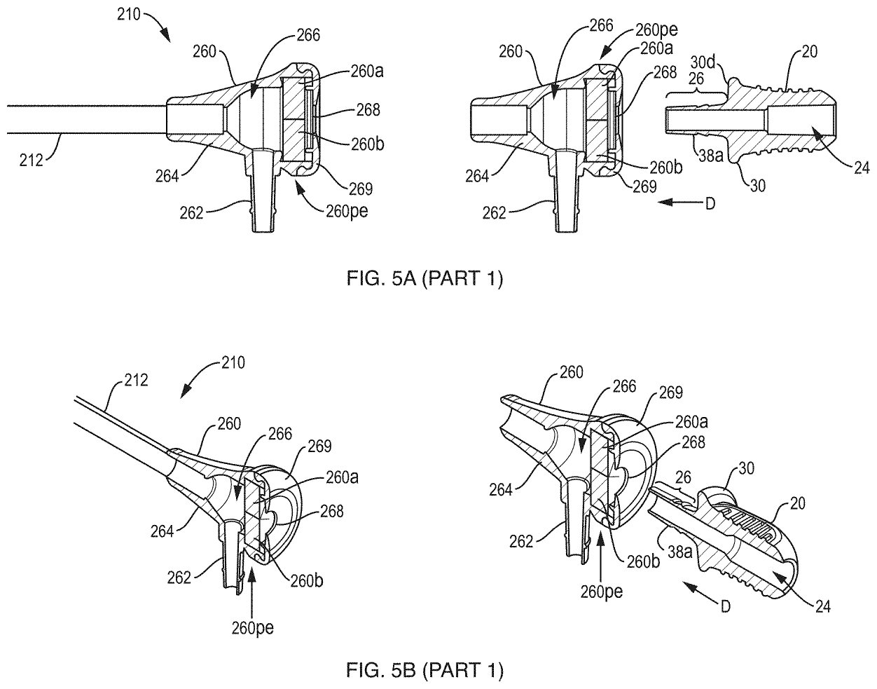

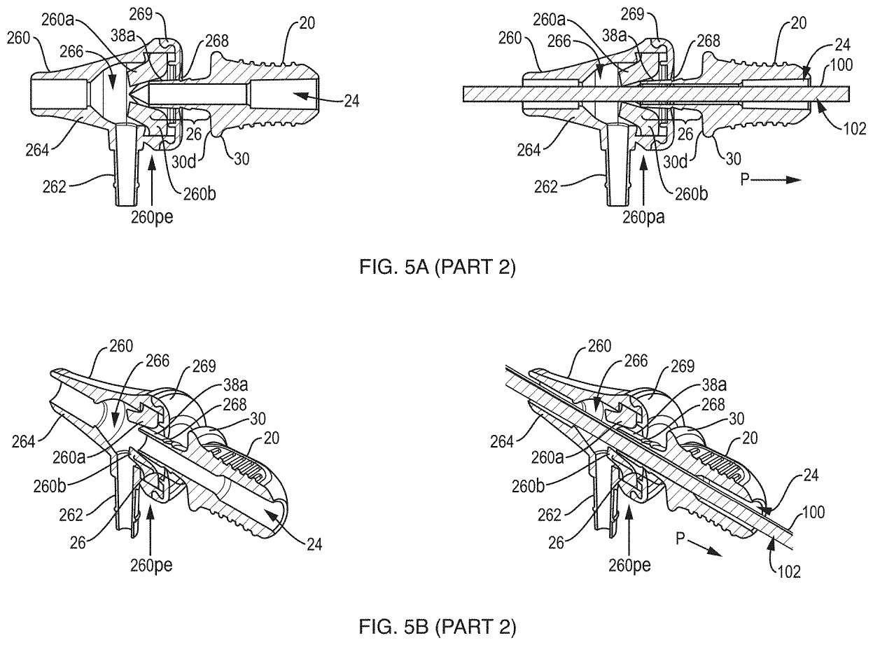

The second hemostatic valve 60 to which the flexible tubing 50 can be coupled can be a conventional or customized valve. For example, the valve 60 can include primary and back-up seals, along with a chamber or inner passageway through which fluid can flow. The port 62 can be provided to provide fluid communication between the valve 60 and the flush line 70. The primary seal can be any type of seal that holds sufficient back pressure to prevent leakage, such as a dome valve or a cross-slit valve. The description, features, and illustrations related to a first hemostatic valve with respect to FIGS. 5A and 5B can also be used in the second hemostatic valve 60, as can other known features of a hemostatic valve. The material or surface of the valve 60 can be provided in a manner that facilitates passage of instruments through the inner passageway while maintaining a seal. Additionally, the second hemostatic valve 60 can optionally include additional features, such as the flush line 70 and control valve 72, to help control the flow of fluid through the extension 10.

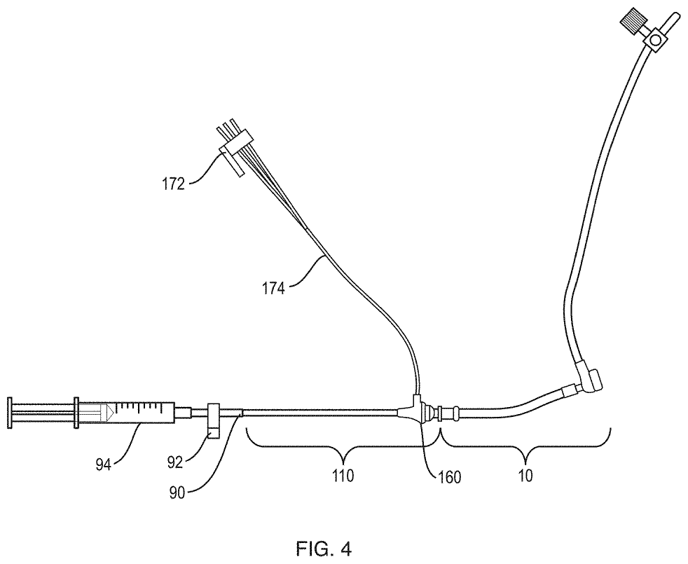

FIG. 4 illustrates the hemostatic valve extension 10 coupled to an introducer sheath 110, sometimes referred to as an access sheath, that includes a first hemostatic valve 160, allowing the extension 10 to be used and/or tested. As shown, the first hemostatic valve 160 is itself connected to tubing 90, which in turn is mated with a valve 92 coupled to a syringe 94, a set-up that can be used, for example, in testing. In other embodiments, as described herein or otherwise known to those skilled in the art, the introducer sheath 110 can be inserted to a surgical site (e.g., the vasculature of a patient). The first hemostatic valve 160 also includes a flush line 170 and control valve 172 (e.g., stopcock) coupled thereto. A person skilled in the art will appreciate the extension 10 can be coupled to a hemostatic valve (e.g., the valve 160) in a variety of set-ups, and thus the illustrated embodiment is by no means limited in that regard. The illustration of FIG. 4 is provided to illustrate one non-limiting instance of how the extension 10 can be associated with an introducer sheath having a first hemostatic valve.

FIGS. 5A and 5B depict the hemostatic connector 20, and thus the extension 10 (other components not illustrated in FIGS. 5A and 5B) as it is secured to a first hemostatic valve 260 of an introducer sheath 210. The introducer sheath 210, which in the illustrated embodiment includes the first hemostatic valve 260 and an access sheath 212 (the term sheath can be used to reference the entire medical device, as well as the sheath component of the medical device) extending distally therefrom, is shown in image (i) of FIGS. 5A and 5B. The sheath 212 is removed from images (ii), (iii), and (iv) of FIGS. 5A and 5B for illustrative purposes. The sheath (e.g., the sheath 212), along with other components of an introducer sheath, can be used in conjunction with the hemostatic connector 20, and the extension 10 more generally. Exemplary introducer sheaths with which the present disclosures can be used include embodiments of the Merit Prelude sheath, the Oscor Adelante sheath, the Teleflex Arrow sheath, and the Terumo Pinnacle sheath. The hemostatic connectors, and the extensions disclosed herein more generally, can be used with other sheaths known or which may be subsequently developed, as well as with other devices that include a hemostatic valve.

As shown in images (ii) of FIGS. 5A and 5B, the hemostatic valve 260 is closed. The valve 260 can include a port 262, a valve body 264, a chamber or passageway 266 formed in the body 264, and an inlet port 268 configured to open and close to provide access to valve 260. A hemostatic valve cap 269 can be mated to a proximal end 260pe of the valve 260, or the cap 269 can be part of the valve 260 itself. As further shown, the hemostatic connector 20 can be advanced in a direction D towards the first hemostatic valve 260. In at least some embodiments, the hemostatic connector 20 can already have a flexible tube (not shown) coupled thereto, as well as a replacement hemostatic valve (not shown) coupled to such a flexible tube and/or other components provided for herein or otherwise known to those skilled in the art. Components such as a flexible tube are not shown to make it easier to focus on other aspects of the extension 10, such as the hemostatic connector 20.

The images (iii) of FIGS. 5A and 5B illustrate the distal end connecting element 26 of the hemostatic connector 20 passing through the hemostatic valve cap 269 and the inlet port 268, thereby causing the first hemostatic valve 260 to be opened. More particularly, leaflets 260a, 260b of the valve 260 are opened, breaking the seal formed by the leaflets 260a, 260b. Any number of leaflets can be used to form the seal. As shown, the first, distal-most barb 38a is seated just distally past the inlet port 268. The distal-most barb 38a can form a seal with a proximal side of that inlet 268 and/or the valve cap 269, creating a sealed passageway between the passageway 266 of the first valve 260 and the passageway 24 of the hemostatic connector 20. When pressed into the body 264 and/or chamber 266 of the first hemostatic valve 260, the hemostatic connector 20 disrupts the existing seal to enable passage of fluids and/or devices through the interface, as shown in the images (iv) of FIGS. 5A and 5B. As described herein, the barbs 38a, 38b of the connector 20 are designed to create a sealed interface with multiple geometries of conventional and/or custom hemostatic valves. While in the illustrated embodiment the flange 30 of the hemostatic connector 20 is spaced a distance apart from the first hemostatic valve 260, in other embodiments the hemostatic connector 20 can be designed such that the distal-facing surface 30d of the flange 30 is flush, or substantially flush, with the first hemostatic valve 260.

Once the hemostatic connector 20, and thus the hemostatic valve extension 10, is properly connected to the first hemostatic valve 260, fluid can flow through the extension 10, including through the hemostatic connector 20, the flexible tube 50, the hemostatic valve 60, and/or the flush line 70 in either direction as desired and/or designed. In the illustrated embodiment, a catheter 100 is passed through the hemostatic connector 20 and the first hemostatic valve 260 to allow fluid 102 to flow through the newly created passageway in which the catheter 100 is disposed, although other medical devices besides catheters can be used. The flow of the fluid through the extension 10 can be controlled, for example by the flush line 70 such that as the control valve 72 is opened, fluid can flow, and when it is closed, the flow of fluid can be stopped.

As mentioned above, once the hemostatic valve extension 10 is in place and operable, the first hemostatic valve 260 can serve as a pass-through such that fluid passes through the first hemostatic valve 260 and into the extension 10. This can be done, for example, by closing a control valve (e.g., the control valve 172 of FIG. 4) associated with a flush line (e.g., the flush line 170 of FIG. 4) coupled to the first hemostatic valve 260 by way of the port 262. Alternatively, fluid can be allowed to flow through both flush lines (e.g., the flush line 170 of FIG. 4 coupled to the port 262 and the flush line 70 of FIG. 1 coupled to the port 62 of the extension 10), for example if both control valves (e.g., the control valves 172, 72 of FIGS. 4 and 1, respectively) are opened. Although the present disclosure describes operating the flush lines by opening and closing control valves, a person skilled in the art will appreciate other ways by which fluid can be controlled through the flush lines to enable flow in either direction if desirable.

Once the procedure is completed, or at least once the need for the hemostatic valve extension 10 is over, the extension 10 can be removed by pulling it away from the first hemostatic valve 260 in an opposite direction P from the insertion direction D. The hemostatic system 10, including the connecting device 20, the tubing 50, and the second hemostatic valve 60, allows the passage of a device sized approximately in the range of about 4 Fr to about 8 Fr to pass through the system without leakage.

Notably, the hemostatic connectors (e.g., the hemostatic connector 20) of the present disclosure use a distal end connecting element (e.g., the distal end connecting element 26, which includes the barbs 38) that includes rotation-independent mating features (e.g., the barbs 38). This means that the mating features are designed such that rotational movement does not impact translational movement along the longitudinal axis L extending through the length of the hemostatic connector 20 (a longitudinal axis extending through the entirety of the length of the passageway 24 of the hemostatic connector 20). Likewise, movement translationally does not require rotational movement. As a result, the hemostatic connector 20 can be coupled to an existing hemostatic valve (e.g., the hemostatic valve 260) without rotating either the hemostatic connector or the existing hemostatic valve. This allows the connector 20 to be inserted into the existing hemostatic valve 260 without undesirable twisting or torqueing of the hemostatic connector 20, and thus components associated therewith (e.g., the flexible tubing 50) and/or undesirable twisting or torqueing of components or other things located distal of the hemostatic connector 20 (e.g., the first hemostatic valve 260, tubing extending therefrom, etc.). The ease of the push-in, pull-out design enabled by the design results in less possible trauma to the patient and/or to components being used in the medical procedure being performed. The present design allows for rotation of the connector 20 without rotating the sheath (e.g., the sheath 210), which is not possible with a twist-in, twist-out design.

More particularly, designs that utilize a twist-in, twist-out design (for instance by use of threads, a screw, and/or a rotation motion) can create the undesirable twisting and torqueing. Such twist-in, twist-out designs are not rotation-independent. Rather, to effect translational movement along the longitudinal axis L, a component with a twist-in, twist-out design necessarily must be rotated to work effectively. Typically when a hemostatic valve extension is to be used, portions of the system that are necessarily rotated when utilizing twist-in, twist-out designs are already disposed in a patient, and thus the twisting and torqueing is, at the very least, uncomfortable to the patient, and may cause undesirable trauma to the patient and/or the components being used in the procedure. Further, in a twist-in, twist-out design, there is less flexibility to make adjustment once a sealed configuration between two components is formed as compared to the present design. In the present design, the rotation-independence of the design allows the hemostatic connector 20 to be rotated without changing whether the seal is maintained because rotation does not move the hemostatic connector 20 translationally along the longitudinal axis L. In a twist-in, twist-out design, however, such rotation cannot be made without causing translational movement along the longitudinal axis L, which in turn can result in the seal being broken. Because rotation is not needed to create engagement, the present design provides additional flexibility to the operator when using the extension 10.

Still further, the present design, as compared to a twist-in, twist-out design, also allows for faster, easier connections to be made, making the procedures go faster and possibly be less susceptible to failure. Additionally, because of the adaptability of the hemostatic connector 20 to be used with multiple types of existing hemostatic valves and related components, all while maintaining tight seals, provides further benefits of the present designs. The rotation-independent design of the extension 10 enhances versatility of the devices and methods with which the extension 10 is used, as well as the ergonomics and the ability to maintain a seal, and does not require unnecessary twisting of components with which the extension 10 is used during the course of a procedure.

The disclosed systems (e.g., the extension 10, components thereof, and related components thereof) can be used in arterial and venous procedures that include the percutaneous introduction of intravascular devices, including, but not limited to, fluoroscopy procedures, angiographies, and angioplasties. This may include interventions in the coronary or the peripheral arteries. Systems disclosed herein can be particularly useful in interventional cardiology procedures that require vascular access on the left side of the body, such as left radial access, left pedal access, left ulnar access, and left femoral access. To use the system, the operator would typically first obtain vascular access and then attach the extension directly to the vascular sheath prior to passing catheters.

The disclosed systems can also be useful in Extracorporeal Membrane Oxygenation (ECMO) Circuits. An ECMO circuit pumps and oxygenates a patient's blood outside of the body and requires a significant amount of tubing and fittings. Hemostatic valves in the circuit are typically used to pass cannulae and catheters into the patient's arterial system. A hemostatic extension system as disclosed herein would increase the versatility of the circuit, negating the need to break apart the circuit for ad hoc tube adjustments. This added flexibility would be valuable for such serious procedure.

Another aspect of the present disclosure provides a method of catheterizing a subject using the disclosed devices or systems. A person skilled in the art will appreciate how these various medical procedures are performed, and thus a step-by-step description of how to perform such methods is unnecessary. The present disclosure contemplates the use of the disclosed devices and systems, and/or components thereof, in conjunction with such medical procedures, and the claiming of such is supported by virtue of the knowledge of a person skilled in the art in conjunction with the present disclosures.

In use, a location where a first hemostatic valve (e.g., the valve 260) is going to be inserted is prepped on the patient, for example by sterilizing the location. A needle can be used to puncture the skin within the sterilized location to access the vasculature. A guidewire can be inserted through the needle and into the vessel in which the procedure is being performed. The needle can be subsequently removed. An introducer sheath (e.g., the introducer sheath 210) can be disposed on the guidewire and advanced distally into the vessel for use therein. As a result, the first hemostatic valve 260 of the introducer sheath 210 can be disposed at a location that is accessible for use with the extensions provided for herein (e.g., the extension 10, and other configurations provided for or otherwise derivable from the present disclosure). A person skilled in the art will appreciate there are other techniques known to those skilled in the art that can be used to provide access to a patient's body and insert an introducer sheath into a vessel.

The extension 10 can be removed from any packaging in which one or more components of the extension is disposed. The extension 10 can be flushed to remove air and/or otherwise ensure its sterility. The extension 10 can then be coupled to the first hemostatic valve 260 by pushing the distal end connecting element 26 of the hemostatic connector 20 into the first hemostatic valve 260, without rotating the distal end connecting element 260 or the first hemostatic valve 260, as described above with respect to FIGS. 5A and 5B. Confirmation that a proper seal was formed between the distal end connecting element 26 and the first hemostatic valve 260 can be confirmed through tactile feedback, auditory feedback, and/or visual feedback. The extension 10 can then be flushed again to remove air and/or otherwise ensure its sterility.

As described above, the extension 10 can then be operated in a manner that allows the first hemostatic valve 260 to be bypassed, and a second hemostatic valve (e.g., the hemostatic valve 60; which may or may not be considered part of the extension), coupled to the hemostatic connector 20 of the extension 10, can be operated in a similar fashion as the first hemostatic valve 260 would have otherwise been operated. Catheters, stents, and other interventional devices can be inserted and manipulated within the pathway formed by the extension 10, the introducer sheath 210 (including the first hemostatic valve 260 by virtue of being a pass-through valve), and the vasculature until the procedure is completed, at which point the devices can be removed from the vasculature, the introducer sheath 210, and the extension 10. The extension 10 can be removed simultaneously with the introducer sheath 210, or it can be separated from the introducer sheath 210 prior to removing the introducer sheath 210.

The devices disclosed herein is typically designed to be disposed of after a single use, although it is conceivable that such devices could be used multiple times if properly reconditioned and/or sterilized in between uses. Those skilled in the art will appreciate that reconditioning of a device can utilize a variety of techniques for cleaning. Use of such techniques, and the resulting reconditioned device, are all within the scope of the present application.

Preferably, the devices described herein will be processed before the procedure. First, a new or used device is obtained and, if necessary, cleaned. The device can then be sterilized. One common way to sterilize new medical devices is to use ethylene oxide. In one sterilization technique, the device is placed in a closed and sealed container, such as a plastic or TYVEK® bag. The container and device are then placed in a field of radiation that can penetrate the container, such as gamma radiation, x-rays, or high-energy electrons. The radiation kills bacteria on the device and in the container. The sterilized device can then be stored in the sterile container. The sealed container keeps the device sterile until it is opened in the medical facility.

It is preferred that the device is sterilized. This can be done by any number of ways known to those skilled in the art including beta or gamma radiation, ethylene oxide, steam, and a liquid bath (e.g., cold soak).

One skilled in the art will readily appreciate that the present disclosure is well adapted to carry out the objects and obtain the ends and advantages mentioned, as well as those inherent therein. Further, one skilled in the art will appreciate further features and advantages of the disclosure based on the above-described embodiments. Accordingly, the disclosure is not to be limited by what has been particularly shown and described, except as indicated by the appended claims. For example, while the extension is primarily described herein as being used in conjunction with extending a reach of an introducer sheath and/or existing hemostatic valve, the extension and related disclosures can be used and/or otherwise adapted for use in providing extensions for other medical devices.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A hemostatic valve extension, comprising:
a hemostatic connector having a body, a distal end connecting element with a rotation-independent mating feature, a proximal end connecting element, and an internal passageway extending through the body from a distal terminal end of the hemostatic connector to a proximal terminal end of the hemostatic connector;
a flexible tube having a distal end and a proximal end, the distal end being coupled to the proximal end connecting element, the flexible tube being in fluid communication with the internal passageway of the hemostatic connector; and
a replacement hemostatic valve coupled to the proximal end of the flexible tube, the replacement hemostatic valve being in fluid communication with the flexible tube,
wherein the rotation-independent mating feature of the distal end connecting element is configured to couple to an existing hemostatic valve without rotating either the distal end connecting element or the existing hemostatic valve.

2. The hemostatic valve extension of claim 1, wherein the rotation-independent mating feature comprises at least one male barb disposed on an outer surface of the distal end connecting element.

3. The hemostatic valve extension of claim 2, wherein the at least one male barb comprises at least two male barbs.

4. The hemostatic valve extension of claim 3, wherein a maximum diameter of a first male barb of the at least two male barbs is different than a maximum diameter of a second male barb of the at least two male barbs.

5. The hemostatic valve extension of claim 4, wherein the outer surface of the distal connecting element is tapered between the first male barb and the second male barb.

6. The hemostatic valve extension of claim 5, wherein the outer surface of the distal connecting element is tapered between the distal terminal end of the hemostatic connector and the first male barb.

7. The hemostatic valve extension of claim 6, wherein the maximum diameter of each of the first and second male barbs is approximately in the range of about 3 millimeters to about 6 millimeters.

8. (canceled)

9. The hemostatic valve extension of claim 7, wherein the maximum diameter of the first male barb is approximately 3.9 millimeters and the maximum diameter of the second male barb is approximately 4.2 millimeters.

10. (canceled)

11. (canceled)

12. The hemostatic valve extension of claim 1, wherein the body further comprises a flange disposed between the distal end connecting element and a proximal portion of the body.

13. (canceled)

14. A hemostatic connector, comprising:
a body;
a distal end connecting element having at least two male barbs disposed on an outer surface thereof, a maximum diameter of a first male barb of the at least two male barbs being different than a maximum diameter of a second male barb of the at least two male barbs;
a proximal end connecting element; and
an internal passageway extending through the body from a distal terminal end of the hemostatic connector to a proximal terminal end of the hemostatic connector.

15. The hemostatic connector of claim 14, wherein the outer surface of the distal connecting element is tapered between the first male barb and the second male barb.

16. The hemostatic connector of claim 15, wherein the outer surface of the distal connecting element is tapered between the distal terminal end of the hemostatic connector and the first male barb.

17. The hemostatic connector of claim 16, wherein the maximum diameter of each of the first and second male barbs is approximately in the range of about 3 millimeters to about 6 millimeters.

18. (canceled)

19. The hemostatic connector of claim 17, wherein the maximum diameter of the first male barb is approximately 3.9 millimeters and the maximum diameter of the second male barb is approximately 4.2 millimeters.

20. (canceled)

21. (canceled)

22. The hemostatic connector of claim 14, wherein the body further comprises a flange disposed between the distal end connecting element and a proximal portion of the body.

23. The hemostatic connector of claim 14, further comprising a flexible tube coupled to the proximal end connecting element, the flexible tube being in fluid communication with the internal passageway of the hemostatic connector.

24. (canceled)

25. (canceled)

26. A method of performing an interventional medical procedure, comprising:
coupling a hemostatic connector to a first hemostatic valve disposed in a patient without rotating either the hemostatic connector or the first hemostatic valve; and
performing one or more interventional medical procedures from a location that is on an opposite side of the patient than where the first hemostatic valve is located, wherein the hemostatic connector is coupled to a flexible tube and the flexible tube is coupled to a second hemostatic valve, the hemostatic connector, the flexible tube, and the second hemostatic valve being in fluid communication with each other, and wherein coupling the hemostatic connector to the first hemostatic valve allows the first hemostatic valve to be bypassed and the second hemostatic valve to be operable to perform one or more medical procedures.

27. The method of claim 26, wherein coupling a hemostatic connector to a first hemostatic valve comprises pushing a distal end connecting element of the hemostatic connector into the first hemostatic valve, forming a seal therebetween.

28. The method of claim 27, wherein the distal end connecting element comprises at least two male barbs disposed on an outer surface thereof, a maximum diameter of a first male barb of the at least two male barbs being different than a maximum diameter of a second male barb of the at least two male barbs to enable the hemostatic connector to be compatible with multiple sizes of hemostatic valves that can be the first hemostatic valve.

29. The method of claim 26, wherein the one or more interventional medical procedures comprise one or more of: arterial procedures that use percutaneous introduction of intravascular devices, cardiology procedures, procedures involving Extracorporeal Membrane Oxygenation Circuits, or catheterizing.
